(19) United States
(12) Patent Application Publication
(10) Pub. No.: US 2016/0102332 A1
(43) Pub. Date: Apr. 14, 2016

(54) METHODS FOR MAKING TARGETED PROTEIN TOXINS BY SORTASE-MEDIATED PROTEIN LIGATION

(71) Applicant: President and Fellows of Harvard College, Cambridge, MA (US)

(72) Inventors: R. John Collier, Wellesley, MA (US); Andrew J. McCluskey, Shrewsbury, MA (US); Bradley L. Pentelute, Cambridge, MA (US)

(21) Appl. No.: 14/727,017

(22) Filed: Jun. 1, 2015

Related U.S. Application Data
(63) Continuation of application No. PCT/US13/72552, filed on Dec. 2, 2013.
(60) Provisional application No. 61/732,526, filed on Dec. 3, 2012.

(51) Int. Cl. C12P 21/00; A61K 47/48

(52) U.S. Cl. CPC C12P 21/00; A61K 47/48261

(57) ABSTRACT

We described novel methods for making targeted protein toxins by sortase-mediated protein ligation. The methods allow for a toxin and receptor-binding ligand to be ligated under mild conditions in vitro, following their expression and purification as single entities. The methods also provide a much more efficient way of making functional targeted fusion toxins compared to recombinant or chemical production of these structures.

### CROSS-REFERENCE TO RELATED APPLICATION

This application is a continuation application under 35 USC §120 of International Application No. PCT/US13/72552, filed Dec. 2, 2013, which claims benefit under 35 U.S.C. §119(e) of U.S. Provisional Application No. 61/732,526 filed Dec. 3, 2012, the contents of which are incorporated herein by reference in their entireties.

### GOVERNMENT SUPPORT

This invention was made with government support under Grant No. AI 022021 awarded by National Institutes of Health. The Government has certain rights in the invention.

### SEQUENCE LISTING

The instant application contains a Sequence Listing which has been submitted in ASCII format via EFS-Web and is hereby incorporated by reference in its entirety. Said ASCII copy, created on Dec. 2, 2013, is named 002806-075511-PCT_SL.txt and is 29,402 bytes in size.

### FIELD OF THE INVENTION

The present invention relates to methods for making targeted protein toxins.

### BACKGROUND

Altering the receptor specificity of a protein toxin to selectively kill distinct cell populations has been an attractive approach in the treatment of cancer. Typically, the receptor binding domain of the toxin is removed or disrupted and chemically or recombinantly linked to receptor-binding ligand. The surrogate ligand functionally replaces the receptor binding domain of the toxin and allows for binding to a specific receptor on the cell surface, where it is subsequently endocytosed and kills the cell.

Original toxin-conjugates were created by chemical reactions (e.g., attaching an antibody and toxin by thiol oxidation). These techniques could result in heterogenous populations of fusion proteins and unconjugated starting materials that could be difficult to fractionate. More recently, the focus has shifted to creating targeted toxins using recombinant DNA technology by simply fusing the protein coding sequences for the toxin and the receptor ligand and expressing the fusion protein as a single entity. However, these fusions are not always expressed and the fusion may render the toxin or ligand inactive as the fusion may not allow for independent folding of the two proteins into active conformations.

### SUMMARY OF THE INVENTION

We describe a new approach and method for making functional targeted protein toxins by sortase-mediated protein ligation. Sortases are enzymes from bacteria that catalyze the cleavage of a short recognition motif with the concurrent formation of a covalent bond between the target protein and an oligoglycine peptide.

We have discovered that this system allows for a toxin and receptor-binding ligand to be ligated under mild conditions in vitro, following their expression and purification as single entities. The method provides a much more efficient way of making functional targeted fusion toxins compared to recombinant or chemical production of these structures.

The novel method of creating targeted toxins by sortase-mediated protein ligation is a significant finding as to our knowledge no toxin variant has been ligated to a receptor ligand using the sortase ligation system to date.

To show that this system provides a better way of making targeted protein toxins, we created targeted, single-chain and binary toxin conjugates by ligating a HER2-specific Affibody or antibody fragment to modified forms of diphtheria toxin and anthrax toxin protective antigen, in which the native receptor binding function has been disrupted. The resulting fusion proteins were able to selectively kill HER2-positive cells, without off-target killing of cells lacking the receptor. The method therefore resulted in proper folding of the functional domains of the fusion protein comprising a toxin and a receptor targeting protein. Thus, we showed that the use of sortase A represents a versatile method to alter the receptor specificity of intracellularly acting toxins and provides an alternative to recombinant expression of single chain toxins.

Accordingly, we provide a novel method for making a protein toxin comprising a receptor-binding ligand, the method comprising the steps of: providing a protein toxin substrate and a C-terminal sortase-recognition motif optionally followed by an affinity epitope that is separated from the toxin by a linker; providing a targeting moiety comprising an N-terminal peptide; and contacting the protein toxin substrate with the targeting ligand with a sortase enzyme.

In some aspects of all the embodiments of the invention, the protein toxin substrate does not comprise its natural receptor binding domain or comprises a non-functional natural receptor binding domain. In other words, one can remove or disrupt the receptor-binding domain of the toxin prior to appending a receptor-targeting ligand.

In aspects where the natural or wild-type toxin receptor binding moiety is not deleted, partially or completely or rendered non-functional by mutating it, one can inhibit toxin receptor-binding by using excess of receptor or receptor mimics. In this approach one saturates the receptor-binding domain with an excess of receptor or receptor-mimic.

In some aspects of all the embodiments of the invention, the targeting moiety is a receptor targeting ligand.

In some aspects of all the embodiments of the invention, the targeting moiety is a cancer cell targeting moiety. For example, targeting moieties or ligands that bind to receptors specifically or more abundantly expressed on cancer cells can be used as the targeting moiety. Such receptors include, for example HER1, HER2, HER3 and HER4, EGF receptors; vascular endothelial growth factor receptors VEGFR-1, VEGFR-2 and VEGFR-3; insulin-like growth factor 1 receptors; fibroblast growth factor receptors; thrombospondin 1 receptors; estrogen receptors; urokinase receptors; progesterone receptors; testosterone receptors; carcinoembryonic antigens; prostate-specific antigens; farnesoid X receptors; transforming growth factor receptors; transferrin receptors; hepatocyte growth factor receptors; or vasoactive intestinal polypeptide receptors 1 and 2.

In some aspects of all the embodiments of the invention, the receptor is HER 2.

In some aspects of all the embodiments of the invention, the receptor targeting ligand is an antibody or an AFFIBODY. For example, an antibody or AFFIBODY targeting HER1, HER2, HER3 and HER4, EGF receptors; vascular endothelial growth factor receptors VEGFR-1, VEGFR-2 and VEGFR-3; insulin-like growth factor 1 receptors; fibroblast growth factor receptors; thrombospondin 1 receptors; estrogen receptors; urokinase receptors; progesterone receptors; testosterone receptors; carcinoembryonic antigens; prostate-specific antigens; farnesoid X receptors; transforming growth factor receptors; transferrin receptors; hepatocyte growth factor receptors; or vasoactive intestinal polypeptide receptors 1 and 2 can be used. In some aspects of all the embodiments of the invention, the targeting moiety is HER2 antibody or HER2 AFFIBODY.

In some aspects of all the embodiments of the invention, the C-terminal sortase recognition motif is a sortase A (SrtA) recognition motif.

In some aspects of all the embodiments of the invention, the sortase A recognition motif is LPXTG (SEQ ID NO: 1), wherein X is any amino acid.

In some aspects of all the embodiments of the invention, the sortase A recognition motif is LPETGG (SEQ ID NO: 2).

In some aspects of all the embodiments of the invention, the C-terminal sortase recognition motif is a sortase B recognition motif.

In some aspects of all the embodiments of the invention, the sortase B recognition motif is NPQTN (SEQ ID NO: 3) or NPKTG (SEQ ID NO: 4).

In some aspects of all the embodiments of the invention, the affinity epitope is selected from a Histidine repeat (His₆) (SEQ ID NO: 5), maltose binding protein (MBP), protein A (ProtA), glutathione 5-transferase (GST), calmodulin binding peptide (CBP), calmodulin, thioredoxin, Strep-tags, hemagglutinin, biotin, FLAG, V5, and c-myc.

In some aspects of all the embodiments of the invention, the linker comprises at least one Glycine-Serine repeat.

In some aspects of all the embodiments of the invention, the linker comprises 1-10 Glycine-Serine repeats (SEQ ID NO: 6).

In some aspects of all the embodiments of the invention, the linker comprises 3 or 4 Glycine-Serine repeats (SEQ ID NO: 7).

In some aspects of all the embodiments of the invention, the N-terminal peptide consists of more than two Glycine residues.

In some aspects of all the embodiments of the invention, the N-terminal peptide consists of 3-10 Glycine residues (SEQ ID NO: 8).

In some aspects of all the embodiments of the invention, the N-terminal peptide consists of five Glycine residues (SEQ ID NO: 9).

In some aspects of all the embodiments of the invention, the protein toxin is an anthrax toxin.

In some aspects of all the embodiments of the invention, the protein toxin is a diphtheria toxin.

In some aspects of all the embodiments of the invention further comprises the step of purifying the toxin that comprises a receptor-binding ligand.

In some aspects of all the embodiments of the invention, the step of purifying comprises sequential Ni²⁺-NTA affinity and size exclusion chromatography.

Another aspect of the invention provides a method for making a protein toxin comprising a receptor-binding ligand, the method comprising the steps of: providing a protein toxin substrate and an N-terminal peptide; providing a targeting moiety and a C-terminal sortase-recognition motif optionally followed by an affinity epitope that is separated from the targeting moiety by a linker; and contacting the protein toxin substrate with the targeting ligand with a sortase enzyme.

### BRIEF DESCRIPTION OF DRAWINGS

FIGS. 1A-1B show one exemplary strategy for altering toxin receptor-specificity by sortase-mediated protein ligation. In FIG. 1A, the protein toxin substrate has a C-terminal sortase recognition motif, for example LPETGG (SEQ ID NO: 2), as used in the example, optionally followed by an affinity epitope, such as for example His6 (SEQ ID NO: 5), separated from the toxin by a Glycine-Serine linker comprising at least one Glycine-Serine motif, for example (GS)3- (SEQ ID NO: 10) or (GS)4-linker (SEQ ID NO: 11). The receptor binding protein (RBP), for example, a receptor targeting ligand, is engineered to contain an N-terminal spacer or tag, which is longer than two Glycine residues, such as 3-10 Glycines (SEQ ID NO: 8), we used in our example an oligoglycine consisting of five Glycines (G5) (SEQ ID NO: 9). RBP is an example of a targeting moiety. In the example, the Sortase enzyme cleaves between the threonine and glycine residues in the recognition sequence of the toxin and the N-terminal oligoglycine on the receptor ligand reacts with the newly created toxin C-terminus to yield a toxin-ligand transpeptidation product. This fusion product can be optionally further purified from the unreacted starting materials, for example, by sequential Ni2+-NTA affinity and size exclusion chromatography. FIG. 1B shows four exemplary fusion proteins created using the strategy outlined in FIG. 1A. FIGS. 1A and 1B disclose “[GS]3-LPETGG-His6” as SEQ ID NO: 45, “G5” as SEQ ID NO: 9, “His6” as SEQ ID NO: 5, “GG-His6” as SEQ ID NO: 13 and “[GS]3-LPETG5” as SEQ ID NO: 46.

FIGS. 2A-2B show another strategy for altering toxin receptor-specificity by sortase-mediated protein ligation. The sortase ligation method may also be adapted to label modified toxins (mTx) at the N terminus by appending a C-terminal sortase recognition motif (LPETGG) (SEQ ID NO: 2) and His6 (SEQ ID NO: 5) affinity tag on the receptor-binding protein (RBP) and an N-terminal oligoglycine peptide on the mTx (FIG. 2A). FIG. 2B shows two exemplary fusion proteins created using the strategy outlined in FIG. 2A. By this strategy two HER2 receptor-targeted protein toxin fusions were created by ligating either a ZHER2 Affibody or 4D5 scFv RBP, to a fragment of Pseudomonas exotoxin A (PE38KDEL) (“KDEL” disclosed as SEQ ID NO: 12). FIGS. 2A and 2B disclose “[GS]3-LPETGG-His6” as SEQ ID NO: 45, “G5” as SEQ ID NO: 9, “His6” as SEQ ID NO: 5, “GG-His6” as SEQ ID NO: 13, “[GS]3-LPETG5” as SEQ ID NO: 46 and “KDEL” as SEQ ID NO: 12.

FIGS. 3A-3C show SDS-PAGE analysis for protein-protein ligation by SrtA. FIG. 3A is an image of SDS-PAGE analysis for the fusion reaction creating mDT-ZHER2, mDT-4D5, mPA-ZHER2, or mPA-4D5 when an evolved form of SrtA (SrtA*) was used as catalyst. The reactions were stopped at the indicated times. Gray arrows indicate the shift in SDS-PAGE mobility for the ligated mTx-RBP fusions, compared with unligated forms (black arrow). FIG. 3B is an image of SDS-PAGE analysis for the fusion reaction creating mDT-ZHER2, mDT-4D5, mPA-ZHER2, or mPA-4D5 when wild type (WT) SrtA was used as catalyst. The reactions were stopped at the indicated times. Gray arrows indicate the shift in SDS-PAGE mobility for the ligated mTx-RBP fusions, compared with unligated forms (black arrow). FIG. 3C is an image of SDS-PAGE analysis for purified fusion products, as visualized by coomassie blue staining FIG. 3C discloses “G5” as SEQ ID NO: 9 and “LPETGG-His6” as SEQ ID NO: 44.

FIGS. 4A-4B show that Sortase creates mPA-ZHER2 and mPA-HER2ScFv fusions that mediate specific killing of HER2-positive cells. Cells were incubated with either mPA-ZHER2 (FIG. 4A) or mPA-HER2ScFv (FIG. 4B) and increasing concentrations of LF_{N}-DTA for 4 hours. Cells were washed and exposed to medium containing [³H]-leucine for 1 hour and protein synthesis was measured by scintillation counting and normalized against untreated cells. Cells expressing high, medium, low, and no detectable levels of HER2 are filled circle, square (open or filled), triangle and diamond respectively. Each point on the curves represents the average of experiments performed in quadruplicate.

FIGS. 5A-5B show that Diphtheria toxin (DT) can be redirected to the HER2 receptor by sortase-mediated protein ligation. A truncated form of DT, lacking its receptor binding domain, DT(386) was covalently linked to either of two HER2-targeted ligands, ZHER2 (FIG. 5A) or an anti-HER2 ScFv (FIG. 5B) and exposed to a panel of tumor cell lines alone. After 24 hr, the medium was removed and cells were washed and exposed to medium supplemented with [³H]-leucine for 1 hour. Protein synthesis was measured by scintillation counting and normalized against untreated cells. Cells expressing high, medium, low, and cells lacking the HER2 receptor are filled circle, square (open or filled), triangle and diamond respectively. Each point on the curves represents the average of four experiments.

FIGS. 6A-6B show that HER2-targeted exotoxin A fusions mediate killing of HER2-positive cells. Cells expressing various levels of HER2 were incubated with increasing concentrations of ZHER2-PE38KDEL (“KDEL” disclosed as SEQ ID NO: 12) (FIG. 6A) or 4D5-PE38KDEL (“KDEL” disclosed as SEQ ID NO: 12) (FIG. 6B) for 24 hours. Cells were washed and exposed to medium containing [3H]-leucine for 1 hour. Protein synthesis was measured by scintillation counting and normalized against untreated cells. Results with cells expressing high, medium, low, or no HER2 receptor are square, circle (filled or open), triangle and diamond respectively. Each point on the curves represents the average of 4 experiments. FIGS. 6A and 6B disclose “KDEL” as SEQ ID NO: 12.

FIGS. 7A-7D show that receptor-redirected protein toxins specifically kill HER2-positive tumor cells in a heterogeneous population. Cells were plated in separate compartments of a chambered slide and incubated at 37° C. The next day, the partition was removed, and the slide was incubated with mPA-ZHER2 plus LF_{N}-DTA (FIG. 7A), mPA-4D5 and LF_{N}-DTA (FIG. 7B), mDT-ZHER2 (FIG. 7C), or mDT-4D5 (FIG. 7D). After 24 hours, cells were incubated for 1 hour with medium supplemented with [3H]-leucine and dissolved in 6 mol/Lguanidine-HCl. The incorporated radiolabel was quantified by scintillation counting and percentage proteinsynthesis was normalized against untreated cells.

FIG. 8 shows competition by ZHER2 and 4D5 for mPA-ZHER2- and mPA-4D5-dependent killing. Cells overexpressing HER2 (BT-474) were exposed to mPA-ZHER2 (solid lines) or mPA-4D5 (broken lines) and LFN-DTA, plus free G5-ZHER2 (“G5” disclosed as SEQ ID NO: 9) (filled symbols) or G5-4D5 (“G5” disclosed as SEQ ID NO: 9) (open symbols). After 4 hours, medium was replaced with medium supplemented with [3H]-leucine for 1 hour. Protein synthesis was measured by scintillation counting and normalized against untreated cells. Each point on the curves represents the average of four experiments.

### DETAILED DESCRIPTION OF THE INVENTION

We describe a new approach for creating targeted protein toxins by sortase-mediated protein ligation.

Sortases are a group of enzymes that catalyze the cleavage of a short recognition motif with the concurrent formation of a covalent bond between the target protein and an oligoglycine peptide.

We show herein, that sortases can be used to make targeted protein toxins where a targeting motif is added to the toxin delivery vehicle enzymatically.

SrtA-based protein fusion is appealing from many perspectives. (i) It can circumvent potential problems in expression and/or folding of recombinantly fused polypeptides into their respective active configurations. Thus, even the individual mPA and 4D5 proteins expressed, folded, and underwent rapid Srt*-mediated fusion to yield a biologically active product. (ii) Srt-based fusion avoids the need to tailor a purification protocol for each individual chimeric protein, as is required when such proteins are produced recombinantly. (iii) Preparation of subsets of pure, appropriately tagged fusion partners opens the possibility of easily preparing large number of fusions (the algebraic product of the numbers of entities in the subsets) for testing. With the protocol described here, in which both the sortase enzyme and the toxin protein substrate carried e.g., a His6 (SEQ ID NO: 5) tag, we were able to remove the enzyme, unreacted toxin protein, and the GGH6 (SEQ ID NO: 13) peptide product with a Ni2+-NTA column. Subsequent removal of unreacted RBP on a size exclusion column yielded the desired chimeric proteins in substantially pure form. (iv) With chimeric toxins in which the individual fusion moieties are nontoxic and only the fusion product displays toxic properties, sortase-based fusion of the purified tagged substrates avoids biosafety issues that may arise in expressing the fused polypeptide in vivo.

We use an evolved sortase A enzyme from Staphylococcus aureus, which had a particularly effective cleavage of its target sequence LPETGG (SEQ ID NO: 2). However sortase enzymes are produced by almost all Gram-positive bacteria and some Gram-negative species. Any of these sortase enzymes could work in the same capacity.

Thus, any sortase with a known recognition sequence can be used in the methods of the invention. In some aspects of all the embodiments, the recognition sequence is added to the C-terminus of the protein toxin substrate. This can be done, for example, by making a recombinant fusion protein based on the protein toxin substrate with a C-terminally added sortase recognition sequence.

In some aspects of all the embodiments, the recognition sequence is added to the C-terminus of the targeting moiety.

For example, sortase A (SrtA) is an enzyme from Staphylococcus aureus that catalyzes the cleavage of a short recognition motif (LPXTG) (SEQ ID NO: 1) with the concurrent formation of a covalent bond between the target protein and an oligoglycine peptide. This system allows for a toxin and receptor-binding ligand to be ligated under mild conditions in vitro, following their expression and purification as single entities. We created targeted, single-chain and binary toxin conjugates by ligating a HER2-specific Affibody or antibody fragment to modified forms of diphtheria toxin and anthrax toxin protective antigen, in which the native receptor binding function has been disrupted. The resulting fusion proteins were able to selectively kill HER2-positive cells, without off-target killing of cells lacking the receptor in both single and mixed cell populations. The use of sortase A represents a versatile method to alter the receptor specificity of intracellularly acting toxins and provides an alternative to recombinant expression of single chain toxins. A receptor-binding ligand is one example of a receptor-binding protein.

In our example method, we used an evolved sortase A enzyme (SrtA*) with a recognition sequence: LPETGG (Chen et al. “A general strategy for the evolution of bond-forming enzymes using yeast display” Proc Natl Acad Sci USA. 2011 Jul. 12; 108(28):11399-404. Epub 2011 Jun. 22, 2011, incorporated herein by reference regarding description of the evolved sortase A enzyme). This sequence provides particularly efficient processing for altering the toxin receptor specificity. This enzyme displays better kinetic properties compared to the wild-type sortase A enzyme. The evolved sortase A enzyme we used in our example has three point mutations (P94S/D160N/K196T) and allows for improved catalytic activity, therefore more efficient conjugation of protein substrates.

In some aspects of all the embodiments of the invention, one does not use the wild-type sortase recognition sequence of LPSTG (SEQ ID NO: 14). In some aspects of all the embodiments of the invention, one does not use the LPSTG (SEQ ID NO: 14) sortase recognition sequence to modify the lethal factor effector protein of anthrax toxin.

In addition to sortase A enzymes and their target sequences, one can also use sortase B enzymes, which function with the same principle but use a different sortase recognition sequence. For example, Staphylococcus aureus and Bacillus anthracis produce sortase B enzymes that recognize NPQTN (SEQ ID NO: 3) and NPKTG (SEQ ID NO: 4) motifs, respectively.

The sortase recognition sequence can be optionally followed by an affinity tag or epitope, which can assist in purifying the protein. Multiple different affinity tags and epitopes are known and any one of them can be used in the methods of the invention. For example, one can use a histidine tag, such as His6 (SEQ ID NO: 5), which contains six histidines (SEQ ID NO: 5); maltose binding protein (MBP); protein A (ProtA); glutathione S-transferase (GST); calmodulin binding peptide (CBP); calmodulin, thioredoxin; Strep-tags; hemagglutinin; biotin; FLAG® octapeptide with DYKDDDDK (SEQ ID NO: 15) (1012 Da) sequence, V5 epitope tag, which is derived from a small epitope (Pk) present on the P and V proteins of the paramyxovirus of simian virus 5; and c-myc. One can also use a toxin containing a free cysteine residue as it would react with a solid support with thiol-reactive groups.

One of ordinary skill in the art can easily select any known affinity tag or epitope. Example tag sequences are shown in Table 1 below.

TABLE 1
Example epitope tags useful in the methods
of the invention

| | | SEQ |
|---|---|---|
| | | ID |
| Tag | Sequence/binding partner | No. |
| His₆ | HHHHHH/antibody/Ni²⁺ or Co²⁺ | 5 |
| (SEQ ID | | |
| NO: 5) | | |
| (5-10 | | |
| histidines | | |
| (SEQ ID | | |
| NO: 16) | | |
| are usually | | |
| used in | | |
| histidine | | |
| tags) | | |
| c-MYC | EQKLISEEDL/antibody | 17 |
| HA | YPYDVPDYA/antibody | 18 |
| VSV-G | YTDIEMNRLGK/antibody | 19 |
| HSV | QPELAPEDPED/antibody | 20 |
| V5 | GKPIPNPLLGLDST/antibody | 21 |
| FLAG ® | DYKDDDDK/antibody | 15 |
| AviTag | GLNDIFEAQKIEWHE | 22 |
| Calmodulin- | KRRWKKNFIAVSAANRFKKISSSGAL | 23 |
| tag | | |
| S-tag | KETAAAKFERQHMDS | 24 |
| SBP-tag | MDEKTTGWRGGHVVEGLAGELEQLRARLEHHP | 25 |
| | QGQREP | |
| Softag 1 | SLAELLNAGLGGS | 26 |
| Xpress tag | DLYDDDDK | 27 |
| pilin-C | TDKDMTITFTNKKDAE | 28 |
| protein | | |
| SpyTag | AHIVMVDAYKPTK | 29 |
| BCCP (Biotin | a protein domain recognized | N/A |
| Carboxyl | by streptavidin | |
| Carrier | | |
| Protein) | | |
| Glutathione- | a protein which binds to | N/A |
| S-transfer- | immobilized glutathione | |
| ase-tag | | |
| Green | a protein which is spontaneously | N/A |
| fluorescent | fluorescent and can be bound by | |
| protein-tag | nanobodies | |
| Maltose | a protein which binds to | N/A |
| binding | amylose agarose | |
| protein-tag | | |
| Strep-tag | a peptide which binds to | N/A |
| | streptavidin or the modified | |
| | streptavidin called streptactin | |
| | (Strep-tag II: WSHPQFEK | |
| | (SEQ ID NO: 30)) | |

The epitope tags or other tags can be used to purify the modified toxin protein using affinity purification.

Affinity purification or chromatography, is a well-known technique for purification of, e.g., recombinantly produced proteins. It is based on an interaction between a tag and its binding partner. For example, a tag can be an epitope tag and the binding partner can be an antibody. Other binding partners can also be used, such as avidin-biotin. The immobile phase is typically a gel matrix, for example, agarose; a linear sugar molecule derived from algae (Voet and Voet, Biochemistry John Wiley and Sons; 1995). Usually the starting point is an undefined heterogeneous group of molecules in solution, such as a cell lysate. The molecule of interest will have a well-known and defined property which can be exploited during the affinity purification process. The process itself can be thought of as an entrapment, with the target molecule becoming trapped on a solid or stationary phase or medium. The other molecules in solution will not become trapped as they do not possess this property. The solid medium can then be removed from the mixture, washed and the target molecule released from the entrapment in a process known as elution.

The term “epitope tag” as used herein refers to tags which can be recognized by an antibody. The term “tag” in general refers to any molecule capable of specific interactions with a target in a principle of “lock-key recognition.” The target and tag will constitute an affinity pair, such as antigen/antibody, enzyme/receptor etc.

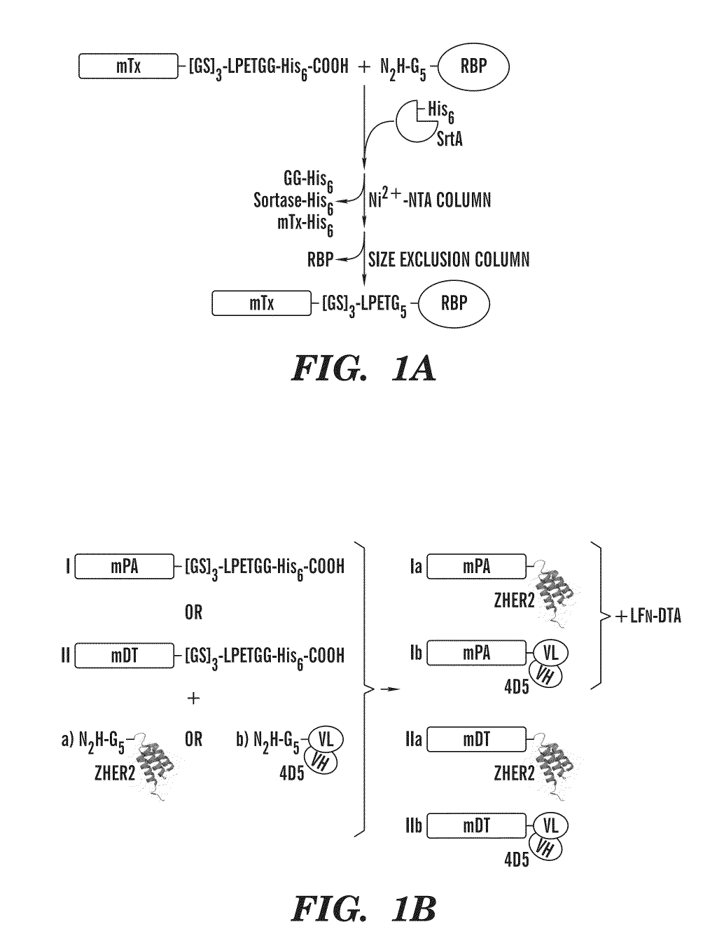

FIG. 1A shows an example of a strategy for altering toxin receptor-specificity by sortase-mediated protein ligation. The protein toxin substrate has a C-terminal sortase recognition motif. As noted before, any sortase recognition motif can be used but in our example we used LPETGG (SEQ ID NO: 2).

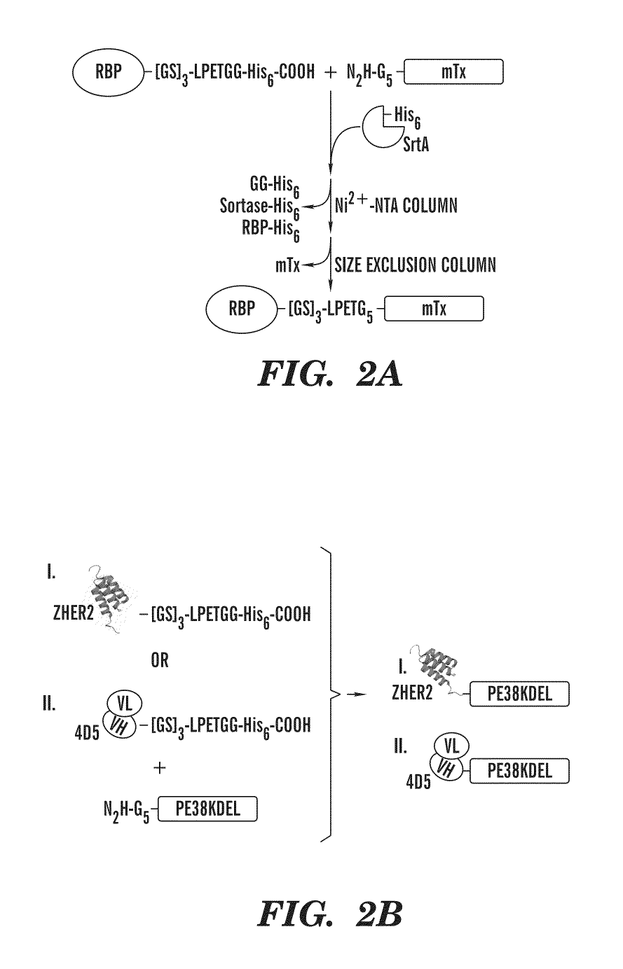

FIG. 2A shows another example of a strategy for altering toxin receptor-specificity by sortase-mediated protein ligation. Thus, for example, the receptor targeting ligand can be engineered to fuse with a C-terminal sortase-recognition motif.

The sortase recognition motif can be optionally followed by an affinity tag or epitope. As noted before, any affinity epitope or affinity tag can be used. We used a six histidine tag (SEQ ID NO: 5) in our example.

We also discovered that it was important to separate the sortase motif from the protein toxin. Addition of the separation peptide is important to provide distance from the toxin C terminus and allowing for efficient display into solution. In our example, we used a repeated glycine/serine peptide-linker (GS)4 (SEQ ID NO: 11) or (GS)3 (SEQ ID NO: 10). However, according to our analyses, the function of providing sufficient separation can be accomplished by any at least two amino acids.

If an affinity tag or epitope is used, it is separated from the toxin by a peptide linker. If no affinity epitope or tag is used, the peptide linker is added after the sortase recognition motif. The linker can be constructed with any at least two amino acids and combinations thereof. One can use for example 2-50 amino acids, 2-40 amino acids, 2-30 amino acids, 2-20 amino acids, 2-10 amino acids and 2-5 amino acids. Typically natural amino acids are used, and they are well known to a skilled artisan. In our example, we used a glycine-serine linker comprising at least one glycine-serine motif (GS). The methods of the invention allow use of one or more GS repeats. For example, one can use at least 2, 3, 4, 5, 6, 7, 8, 9, 10, 11, 12, 13, 14, or at least 15 GS repeats (SEQ ID NO: 31). For example 1-20 (SEQ ID NO: 32), 1-15 (SEQ ID NO: 33), 1-10 (SEQ ID NO: 34), 1-5 (SEQ ID NO: 35) GS repeats can be used. In our examples, we have used (GS)3- (SEQ ID NO: 10) and (GS)4-linker (SEQ ID NO: 11).

In some aspects of all the embodiments, the receptor targeting ligand is separately engineered to contain an N-terminal peptide, typically an oligoglycine, which should be longer than two glycine residues. One can use at least 3, 4, 5, 6, 7, 8, 9, 10, 11, 12, 13, 14, and at least 15 residues long oligoglycines (SEQ ID NO: 47), for example 2-20 (SEQ ID NO: 36), 2-15 (SEQ ID NO: 37), 2-10 (SEQ ID NO: 38) residue long oligoglycines. In our example, we used an oligoglycine consisting of five glycine residues (G5) (SEQ ID NO: 9).

In our example, the sortase A enzyme cleaves between the threonine and glycine residues in the recognition sequence of the toxin and the N-terminal oligoglycine on the receptor ligand reacts with the newly created toxin C-terminus to yield a toxin-ligand transpeptidation fusion product. As discussed above, all sortases work with essentially the same principle and cut at their recognition sites respectively.

This fusion product can be optionally further purified from the unreacted starting materials. The purification can be performed for example with any known affinity and/or size exclusion chromatography. We used, for example, sequential Ni²⁺-NTA affinity and size exclusion chromatography, but any well-known protein purification technique can be used.

In some aspects of all the embodiments, the receptor targeting ligand is separately engineered to fuse with a C-terminal sortase-recognition motif optionally followed by an affinity epitope that is separated from the receptor targeting ligand by a linker.

In the methods of the present invention, one can use a number of different toxins as the delivery vehicle.

The natural toxin receptor recognition site can either be completely or partially deleted or mutated so that it is inoperative. The toxin can thereby be directed to any desired receptor using a targeted ligand.

Toxins according to the invention can include, any toxin that comprises a mechanism to bind a cellular receptor can be used. Table 2 sets forth examples of toxins useful in the methods of the invention.

TABLE 2
Examples of bacterial toxins

| Toxin | Arrangement of subunits A and B in the toxin |
|---|---|
| Cholera toxin | (A-5B) wherein subunits A and B are synthesized separately |
| | and associated by noncovalent bonds; 5B indicates that the |
| | binding domain is composed of 5 identical subunits. |
| Diphtheria toxin | (A/B) wherein subunit domains A and B are of a single |
| | protein that may be separated by proteolytic cleavage. |
| Pertussis toxin | (A-5B) wherein subunits A and B are synthesized separately |
| | and associated by noncovalent bonds; 5B indicates that the |
| | binding (B) domain is composed of 5 identical subunits. |
| E. coli heat-labile toxin LT | (A-5B) wherein subunits A and B are synthesized separately |
| | and associated by noncovalent bonds; 5B indicates that the |
| | binding domain is composed of 5 identical subunits. |
| Shiga toxin | (A/5B) wherein subunit domains A and B are of a single |
| | protein that may be separated by proteolytic cleavage; 5B |
| | indicates that the binding domain is composed of 5 identical |
| | subunits. |
| Pseudomonas Exotoxin | A (A/B) wherein subunit domains A and B are of a single |
| | protein that may be separated by proteolytic cleavage. |
| Botulinum toxin | (A/B) subunit domains are of a single protein that may be |
| | separated by proteolytic cleavage |
| Tetanus toxin | (A/B) wherein subunit domains A and B are of a single |
| | protein that may be separated by proteolytic cleavage |
| Anthrax toxin Lethal Factor | (A2 + B) wherein subunits synthesized and secreted as |
| | separate protein subunits that interact at the target cell |
| | surface |
| Bordetella pertussis AC toxin | (A/B) subunit domains are of a single protein that may be |
| | separated by proteolytic cleavage |
| Bacillus anthracis EF | (A1 + B) wherein subunits synthesized and secreted as |
| | separate protein subunits that interact at the target cell |
| | surface |

Uses of various bacterial toxins for delivery of bioactive molecules has been described, e.g., in WO 2012/096926, which is incorporated herein by reference in its entirety for teaching the toxin delivery systems.

Sequences for all these toxins are well known and available in public databases. Anyone with ordinary skill in the art can manipulate the toxin sequences according to the teachings of this invention to disable the natural toxin receptor binding site by deleting, partially or completely, or mutating the binding site to abolish the natural toxin receptor ligand part if desired.

Bacterial toxin B components, in general, can be used to deliver bioactive moieties into the cytosol of the cells when the bioactive moiety is attached to the A component or a surrogate A component of the bacterial toxin, as long as the bioactive moiety unfolds correctly (if such is required for activity) during translocation. In addition to the anthrax B component, PA, the B components of Clostridium perfringens toxins (alpha, beta, epsilon, iota), C. botulinum C2 toxin, and C. spiroforme Iota-like toxins can be used as described herein.

A bioactive peptide or cytotoxic domain can be attached to an A component of the binary system, such as the nontoxic PA-binding domain of LF (LF_{N}), and the fusion protein thus formed passes through the pore into the cytosol of a cell. See PCT US2012/20731. Cytotoxic domains can be derived from shiga toxin, shiga-like toxin 1 and 2, ricin, abrin, gelonin, pokeweed antiviral protein, saporin, trichsanthin, pepcin, maize RIP, alpha-sarcin, Clostridium perfringens epsiolon toxin, Botulinum neurotoxins, Staphylococcus enterotoxins, difficile toxins, pertussis toxins, or pseudomonas exotoxins.

The actions of the binary toxins depend on their ability to bind to one or more cell-surface receptors. Anthrax toxin acts by a sequence of events that begins when the Protective-Antigen (PA) moiety of the toxin binds to either of two cell-surface proteins, ANTXR1 and ANTXR2, and is proteolytically activated. The activated PA self-associates to form oligomeric pore precursors, which, in turn, bind the enzymatic moieties of the toxin and transport them to the cytosol. More specifically, the PA63 prepore binds up to three or four molecules of LF, forming complexes that are then endocytosed. Upon acidification of the endosome, protective antigen prepore undergoes a conformational rearrangement to form a membrane-spanning, ion-conductive pore, which transports lethal factor from the endosome to the cytosol. LF_{N}, the N-terminal domain of lethal factor, has nanomolar binding affinity for the pore, and this domain alone can be used for translocation of chemical moieties. Additionally, small positively charged peptide segments that mimic LF_{N} can be used to aid in translocating these molecules through PA pore. These mimics may be composed of at least one non-natural amino acid. See PCT US2012/20731. Engineered binary toxin B components, such as PA fusion proteins with altered receptor specificity, are useful in biological research and have practical applications, including perturbation or ablation of selected populations of cells in vivo.

We have previously described a genetically modified PA, carrying a double mutation into domain 4 of PA to ablate its native receptor-binding function and fused epidermal growth factor (EGF) to the C terminus of the mutated protein. The resulting fusion protein transported enzymatic effector proteins into a cell line that expressed the EGF receptor (A431 cells), but not into a line lacking this receptor (CHO-K1 cells). Addition of excess free EGF blocked transport of effector proteins into A431 cells via the fusion protein, but not via native PA. Additionally, fusing the diphtheria toxin receptor-binding domain to the C terminus of the mutated PA channeled effector-protein transport through the diphtheria toxin receptor. The modified PA domain has been described in provisional application No. 61/602,218, filed on Feb. 23, 2012, which is incorporated herein by reference in its entirety.

Briefly, two mutations, N682A and D683A, were introduced into PA to ablate its native receptor-binding function (Rosovitz et al., 278 J. Biol. Chem. 30936 (2003)), and the mutated protein (mPA) was expressed in E. coli BL21 (DE3). The purified product failed to promote entry of LF_{N}-DTA into either CHO-K1 cells or A431 cells at the highest concentration tested (10 nM), as measured by the inhibition of protein synthesis in the presence of LF_{N}-DTA. LF_{N}-DTA is a fusion between LF_{N}, the N-terminal PA63-binding domain of LF, and DTA, the catalytic domain of diphtheria toxin. See PCT US2012/20731. The DTA moiety catalyzes the ADP-ribosylation of eukaryotic elongation factor 2 (eEF-2) within the cytosol, blocking protein synthesis and causing cell death. Collier & Cole, 164 Science 1179 (1969); Collier, 25 J. Mol. Biol. 83 (1967).

The natural toxin receptor binding domain can also be partially or completely deleted. While the sequences for these domains are diverse, a skilled artisan knows that the binding domains are typically contained in the B subunit.

In any aspect of the invention, one can add to the toxin any useful “targeting moiety.” For example, ligands that bind to receptors specifically or more abundantly expressed on cancer cells can be used as the targeting moiety. Such receptors include, for example HER1, HER2, HER3 and HER4, EGF receptors; vascular endothelial growth factor receptors VEGFR-1, VEGFR-2 and VEGFR-3; insulin-like growth factor 1 receptors; fibroblast growth factor receptors; thrombospondin 1 receptors; estrogen receptors; urokinase receptors; progesterone receptors; testosterone receptors; carcinoembryonic antigens; prostate-specific antigens; farnesoid X receptors; transforming growth factor receptors; transferrin receptors; hepatocyte growth factor receptors; or vasoactive intestinal polypeptide receptors 1 and 2.

The “targeting moiety” can be, e.g., an AFFIBODY or an antibody that binds to a specific target, such as a receptor, like HER1, HER2, HER3 and HER4 EGF receptors; vascular endothelial growth factor receptors VEGFR-1, VEGFR-2 and VEGFR-3; insulin-like growth factor 1 receptors; fibroblast growth factor receptors; thrombospondin 1 receptors; estrogen receptors; urokinase receptors; progesterone receptors; testosterone receptors; carcinoembryonic antigens; prostate-specific antigens; farnesoid X receptors; transforming growth factor receptors; transferrin receptors; hepatocyte growth factor receptors; or vasoactive intestinal polypeptide receptors 1 and 2. The targeting moiety can also be a receptor ligand. Other useful targeting moieties are nucleic acids, such as aptamers, and antibody mimetics, such as Affilins, affitins, anticalins, avimers, DARPins, Fynomers, Kunitz domain peptides, and monobodies.

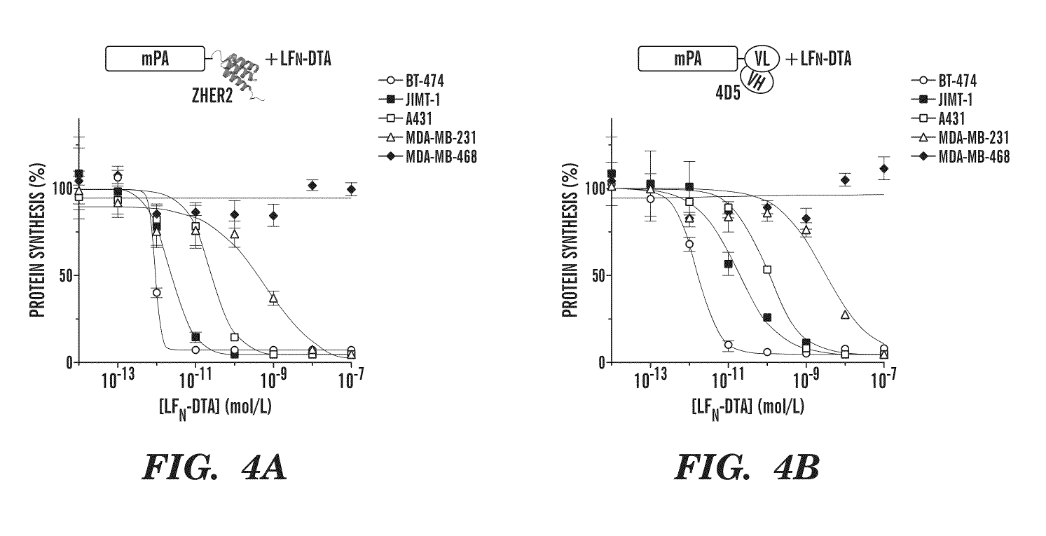

For example, FIGS. 4A and 4B show that Sortase creates mPA-ZHER2 and mPA-HER2ScFv fusions that mediate specific killing of HER2-positive cells. Cells were incubated with either mPA-ZHER2 (FIG. 4A) or mPA-HER2ScFv (FIG. 4B) and increasing concentrations of LF_{N}-DTA for 4 hours. Cells were washed and exposed to medium containing [³H]-leucine for 1 hour and protein synthesis was measured by scintillation counting and normalized against untreated cells. Cells expressing high, medium, low, and no detectable levels of HER2 are filled circle, square (open or filled), triangle and diamond respectively. Each point on the curves represents the average of experiments performed in quadruplicate.

Example of an mPA amino acid sequence for the methods of the invention is provided below. This sequence was used in the examples of the invention.

(SEQ ID NO: 39)
E V K Q E N R L L N E S E S S S Q G L L G Y Y
F S D L N F Q A P M V V T S S T T G D L S I P
S S E L E N I P S E N Q Y F Q S A I W S G F I
K V K K S D E Y T F A T S A D N H V T M W V D
D Q E V I N K A S N S N K I R L E K G R L Y Q
I K I Q Y Q R E N P T E K G L D F K L Y W T D
S Q N K K E V I S S D N L Q L P E L K Q K S S
N S R K K R S T S A G P T V P D R D N D G I P
D S L E V E G Y T V D V K N K R T F L S P W I
S N I H E K K G L T K Y K S S P E K W S T A S
D P Y S D F E K V T G R I D K N V S P E A R H
P L V A A Y P I V H V D M E N I I L S K N E D
Q S T Q N T D S Q T R T I S K N T S T S R T H
T S E V H G N A E V H A S F F D I G G S V S A
G F S N S N S S T V A I D H S L S L A G E R T
W A E T M G L N T A D T A R L N A N I R Y V N
T G T A P I Y N V L P T T S L V L G K N Q T L
A T I K A K E N Q L S Q I L A P N N Y Y P S K
N L A P I A L N A Q D D F S S T P I T M N Y N
Q F L E L E K T K Q L R L D T D Q V Y G N I A
T Y N F E N G R V R V D T G S N W S E V L P Q
I Q E T T A R I I F N G K D L N L V E R R I A
A V N P S D P L E T T K P D M T L K E A L K I
A F G F N E P N G N L Q Y Q G K D I T E F D F
N F D Q Q T S Q N I K N Q L A E L N A T N I Y
T V L D K I K L N A K M N I L I R D K R F H Y
D R N N I A V G A D E S V V K E A H R E V I N
S S T E G L L L N I D K D I R K I L S G Y I V
E I E D T E G L K E V I N D R Y D M L N I S S
L R Q D G K T F I D F K K Y A A K L P L Y I S
N P N Y K V N V Y A V T K E N T I I N P S E N
G D T S T N G I K K I L I F S K K G Y E I G

The amino acid sequence for ZHER2 Affibody is as follows: V D N K F N K E M R N A Y W E I A L L P N L N N Q Q K R A F I R S L Y D D P S Q S A N L L A E A K K L N D A Q A P K (SEQ ID NO: 40).

An example of a HER2 Single chain antibody fragment amino acid sequence as used in the examples is as follows: D I Q M T Q S P S S L S A S V G D R V T I T C R A S Q D V N T A V A W Y Q Q K P G K A P K L L I Y S A S F L Y S G V P S R F S G S R S G T D F T L T I S S L Q P E D F A T Y Y C Q Q H Y T T P P T F G Q G T K V E I K R T P S H N S H Q V P S A G G P T A N S G T S G S E V Q L V E S G G G L V Q P G G S L R L S C A A S G F N I K D T Y I H W V R Q A P G K G L E W V A R I Y P T N G Y T R Y A D S V K G R F T I S A D T S K N T A Y L Q M N S L R A E D T A V Y Y C S R W G G D G F Y A M D Y W G Q G T L V T V S S (SEQ ID NO: 41).

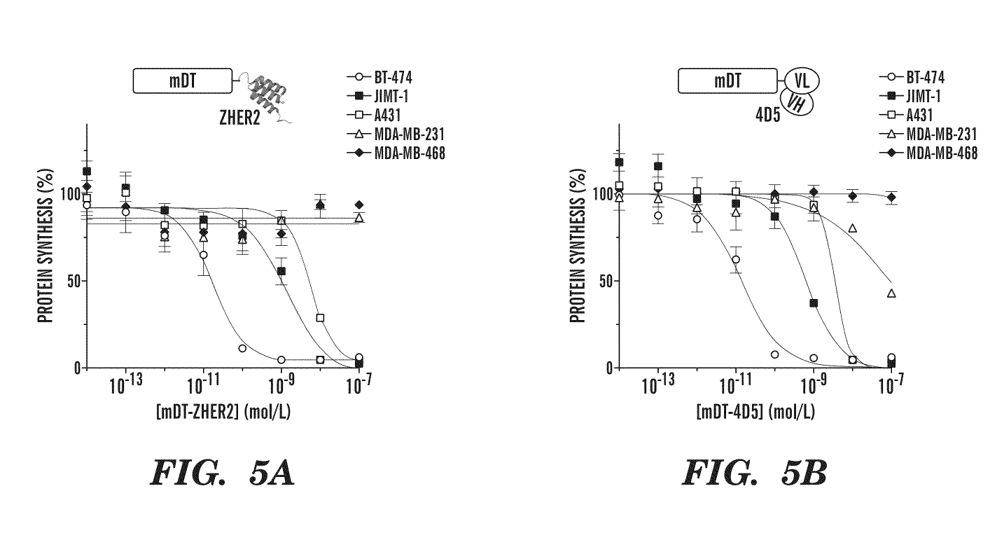

FIGS. 5A and 5B show that Diphtheria toxin (DT) can be redirected to the HER2 receptor by sortase-mediated protein ligation. A truncated form of DT, lacking its receptor binding domain, DT(386) was covalently linked to either of two HER2-targeted ligands, ZHER2 (FIG. 5A) or an anti-HER2 ScFv (FIG. 5B) and exposed to a panel of tumor cell lines alone. After 24 hr, the medium was removed and cells were washed and exposed to medium supplemented with [³H]-leucine for 1 hour. Protein synthesis was measured by scintillation counting and normalized against untreated cells. Cells expressing high, medium, low, and cells lacking the HER2 receptor are filled circle, square (open or filled), triangle and diamond respectively. Each point on the curves represents the average of four experiments.

An example of a modified DT amino acid sequence for the methods of the invention is provided below. This sequence was used in the examples.

```
(SEQ ID NO: 42)
                10 20 30 40
GADDVVDSSK SFVMENFSSY HGTKPGYVDS IQKGIQKPKS
                50 60 70 80
GTQGNYDDDW KGFYSTDNKY DAAGYSVDNE NPLSGKAGGV
                90 100 110 120
VKVTYPGLTK VLALKVDNAE TIKKELGLSL TEPLMEQVGT
              130 140 150 160
EEFIKRFGDG ASRVVLSLPF AEGSSSVSYI NNWEQAKALS
              170 180 190 200
VELEINFETR GKRGQDAMYE YMAQACAGNR VRRSVGSSLS
              210 220 230 240
CINLDWDVIR DKTKTKIESL KEHGPIKNKM SESPNKTVSE
              250 260 270 280
EKAKQYLEEF HQTALEHPEL SELKTVTGTN PVFAGANYAA
              290 300 310 320
WAVNVAQVID SETADNLEKT TAALSILPGI GSVMGIADGA
              330 340 350 360
VHHNTEEIVA QSIALSSLMV AQAIPLVGEL VDIGFAAYNF
              370 380
VESIINLFQV VHNSYNRPAY SPGHKT
```

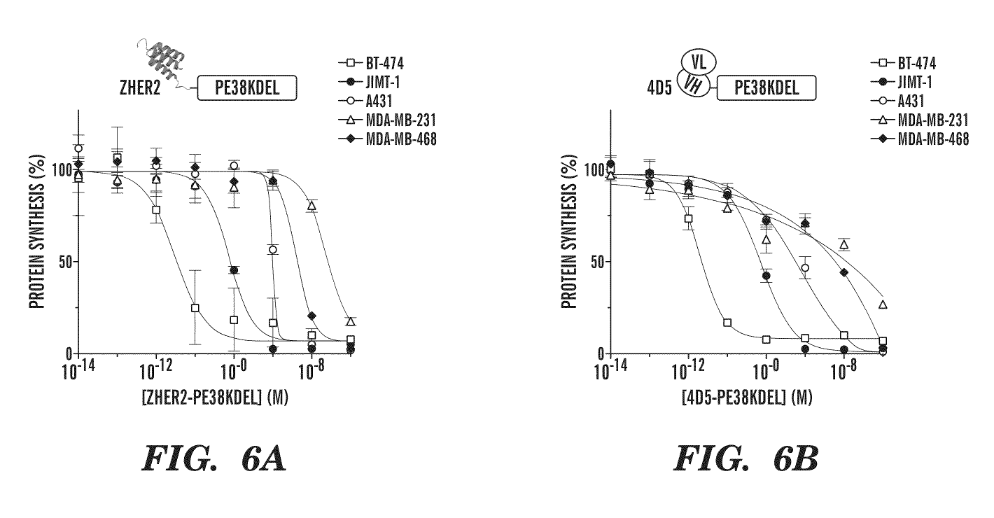

FIGS. 6A and 6B show that HER2-targeted exotoxin A fusions mediate killing of HER2-positive cells. Cells expressing various levels of HER2 were incubated with increasing concentrations of ZHER2-PE38KDEL (“KDEL” disclosed as SEQ ID NO: 12) (FIG. 6A) or 4D5-PE38KDEL (“KDEL” disclosed as SEQ ID NO: 12) (FIG. 6B) for 24 h. Cells were washed and exposed to medium containing [3H]-leucine for 1 h. Protein synthesis was measured by scintillation counting and normalized against untreated cells. Results with cells expressing high, medium, low, or no HER2 receptor are square, circle (filled or open), triangle and diamond respectively. Each point on the curves represents the average of 4 experiments.

An example of a modified Pseudomonas exotoxin A (PE38KDEL) (“KDEL” disclosed as SEQ ID NO: 12) amino acid sequence for the methods of the invention is provided below. This sequence was used in the examples.

```
(SEQ ID NO: 43)
                10 20 30 40
GGSLAALTAH QACHLPLETF TRHRQPRGWE QLEQCGYPVQ
                50 60 70 80
RLVALYLAAR LSWNQVDQVI RNALASPGSG GDLGEAIREQ
                90 100 110 120
PEQARLALTL AAAESERFVR QGTGNDEAGA ANGPADSGDA
              130 140 150 160
LLERNYPTGA EFLGDGGDIS FSTRGTQNWT VERLLQAHRQ
              170 180 190 200
LEERGYVFVG YHGTFLEAAQ SIVFGGVRAR SQDLDAIWRG
              210 220 230 240
FYIAGDPALA YGYAQDQEPD ARGRIRNGAL LRVYVPRSSL
              250 260 270 280
PGFYRTGLTL AAPEAAGEVE RLIGHPLPLR LDAITGPEEE
              290 300 310 320
GGRLETILGW PLAERTVVIP SAIPTDPRNV GGDLDPSSIP
              330 340
DKEQAISALP DYASQPGKPP KDEL
```

We discovered that the success of the method depends on several factors, including: (i) structure/properties of the two proteins of interest, (ii) composition of the sortase recognition motif, (iii) distance of the sortase recognition motif is from the C terminus of the protein, and (iv) variant of sortase enzyme used. We discovered that applying this technology to fusing a toxin to a receptor-targeting ligand also requires that the fusion not hinder the ability of the toxin or the targeting ligand to remain in active conformation. We surprisingly found, that using the parameters discussed above, one was able to create functional targeted toxins.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents. Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

It is noted that methods comprising the indicated steps are generally contemplated. However, also methods consisting essentially of the indicated steps are contemplates. In some embodiments, methods consisting of the indicated steps are contemplated. The term “comprising” is used in its open-ended meaning indicating that additional steps can be included. The term “consisting essentially of” is used to indicate that the essential steps are indicated, but that steps that do not provide a meaningful or substantial change to the method, such as purification or buffer changing steps performed between the indicated steps, can still be included. The term “consisting of” is intended as a closed term, to indicate that the claim only includes the indicated steps.

Some embodiments of the invention are listed in the following paragraphs:

1. A method for making a protein toxin comprising a receptor-binding ligand, the method comprising the steps of:
a. providing a protein toxin substrate and a C-terminal sortase-recognition motif optionally followed by an affinity epitope that is separated from the toxin by a linker;
b. providing a targeting moiety comprising an N-terminal peptide; and
c. contacting the protein toxin substrate of step (a) with the targeting moiety of step (b) with a sortase enzyme.
   2. The method of paragraph 1, wherein the protein toxin substrate does not comprise its natural receptor binding domain or comprises a non-functional natural receptor binding domain.
   3. The method of paragraph 1, wherein the targeting moiety is a receptor targeting ligand.
   4. The method of paragraph 1, wherein the receptor is HER2.
   5. The method of paragraph 3, wherein the receptor targeting ligand is a HER2 antibody or HER2 AFFIBODY.
   6. The method of any of the preceding paragraphs, wherein the C-terminal sortase recognition motif is a sortase A recognition motif
   7. The method of paragraph 6, wherein the sortase A recognition motif is LPXTG (SEQ ID NO: 1), wherein X is any amino acid.
   8. The method of paragraph 7, wherein the sortase A recognition motif is LPETGG (SEQ ID NO: 2).
   9. The method of paragraphs 1-5, wherein the C-terminal sortase recognition motif is a sortase B recognition motif
   10. The method of paragraph 9, wherein the sortase B recognition motif is NPQTN (SEQ ID NO: 3) or NPKTG (SEQ ID NO: 4).
   11. The method of any of the preceding paragraphs, wherein the affinity epitope is selected from a Histidine repeat (His₆) (SEQ ID NO: 5), maltose binding protein (MBP), protein A (ProtA), glutathione 5-transferase (GST), calmodulin binding peptide (CBP), calmodulin, thioredoxin, Strep-tags, hemagglutinin, biotin, FLAG, V5, and c-myc.
   12. The method of any of the preceding paragraphs, wherein the linker comprises at least one Glycine-Serine repeat.
   13. The method of paragraph 12, wherein the linker comprises 1-10 Glycine-Serine repeats (SEQ ID NO: 6).
   14. The method of paragraph 13, wherein the linker comprises 3 or 4 Glycine-Serine repeats (SEQ ID NO: 7).
   15. The method of any of the preceding paragraphs, wherein the N-terminal peptide consists of more than two Glycine residues.
   16. The method of paragraph 15, wherein the N-terminal peptide consists of 3-10 Glycine residues (SEQ ID NO: 8).
   17. The method of paragraph 16, wherein the N-terminal peptide consist of five Glycine residues (SEQ ID NO: 9).
   18. The method of any of the preceding paragraphs, wherein the protein toxin is an anthrax toxin.
   19. The method of any of the preceding paragraphs, wherein the protein toxin is a diphtheria toxin.
   20. The method of any of the preceding paragraphs, further comprising the step of purifying the toxin that comprises a receptor-binding ligand.
   21. The method of paragraph 20, wherein the step of purifying comprises sequential Ni2+-NTA affinity and size exclusion chromatography.
   22. A method for making a protein toxin comprising a receptor-binding ligand, the method comprising the steps of:
d. providing a protein toxin substrate and a N-terminal peptide;
e. providing a targeting moiety comprising an C-terminal sortase-recognition motif optionally followed by an affinity epitope that is separated from the toxin by a linker; and
f. contacting the protein toxin substrate of step (d) with the targeting moiety of step (e) with a sortase enzyme.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1

### Receptor-Directed Chimeric Toxins Created by Sortase-Mediated Protein Fusion

Chimeric protein toxins that act selectively on cells expressing a designated receptor may serve as investigational probes and/or antitumor agents. Here, we report use of the enzyme sortase A (SrtA) to create four chimeric toxins designed to selectively kill cells bearing the tumor marker HER2. We first expressed and purified: (i) a receptor recognition-deficient form of diphtheria toxin that lacks its receptor-binding domain and (ii) a mutated, receptor-binding-deficient form of anthrax-protective antigen. Both proteins carried at the C terminus the sortase recognition sequence LPETGG (SEQ ID NO: 2) and a H6 (SEQ ID NO: 5) affinity tag. Each toxin protein was mixed with SrtA plus either of two HER2-recognition proteins—a single-chain antibody fragment or an Affibody—both carrying an N-terminal G5 tag (SEQ ID NO: 9). With wild-type SrtA, the fusion reaction between the toxin and receptor-recognition proteins approached completion only after several hours, whereas with an evolved form of the enzyme, SrtA*, the reaction was virtually complete within 5 minutes. The four fusion toxins were purified and shown to kill HER2-positive cells in culture with high specificity. Sortase-mediated ligation of binary combinations of diverse natively folded proteins offers a facile way to produce large sets of chimeric proteins for research and medicine.

Materials and Methods.

Oligonucleotides were synthesized by Integrated DNA Technologies. A plasmid encoding the gene sequence for anti-HER2 4D5 scFv (4D5) was received from Gregory Poon (Washington State University, Pullman, Wash.). The wild-type (WT) SrtA and SrtA* expression plasmids were supplied by Brad Pentelute (MIT, Cambridge, Mass.). All chemicals were from Sigma-Aldrich, unless otherwise stated. The A431 cell line was from American Type Culture Collection (cat. no. CCL-1555) and the JIMT-1 cell line was from AddexBio (cat. no. C0006005). BT-474 and MDA-MB-468 cell lines were provided by Jean Zhao (Dana-Farber Cancer Institute, Boston, Mass.) and MDA-MB-231 line by Gregory Poon. Fluorescence-activated cell sorting (FACS) validated HER2 receptor levels. Cells were frozen upon receipt and only low passage number cells were used.

A431 and JIMT-1 cells were maintained in Dulbecco's modified Eagle medium (DMEM) supplemented with 10% fetal calf serum (FCS), 500 U/mL penicillin G and streptomycin sulfate (Invitrogen). All other cell lines were grown in RPMI medium (Invitrogen) supplemented with 10% FCS, 500 U/mL penicillin G and streptomycin sulfate.

mDT (residues 1-387 of diphtheria toxin) was cloned into the petSUMO vector (Invitrogen) with a C-terminal glycine-serine repeat ([GS]3) (SEQ ID NO: 10) linker, SrtA recognition motif (LPETGG) (SEQ ID NO: 2), and hexa-histidine tag (SEQ ID NO: 5), following the standard procedures. mPA, harboring a double mutation (N682A/D683A), was created as described previously (Mechaly et al., mBio 2012, 3, pii:e00088-12; Rosovitz et al., J. Biol. Chem. 2003, 278, 30936-30944) and cloned into the pet22b vector (Novagen) with the same C-terminal [GS]3-linker (SEQ ID NO: 10), SrtA recognition peptide, and His6-tag (SEQ ID NO: 5). LPETGG (SEQ ID NO: 2) was chosen as the SrtA recognition motif because SrtA more rapidly turns over substrates with a G in the P2′ position (Pritz et al, J. Org. Chem. 2007, 72. 3909-0912). Aminoglycine pentapeptides (G5) (SEQ ID NO: 9) were recombinantly fused to RBPs: a HER2-specific Affibody, ZHER2:342 (abbreviated ZHER2), and an anti-HER2 scFv (termed 4D5) containing a 24-amino acid peptide linker between the VL and VH domain (Tang et al., J. Biol. Chem. 1996, 271, 15682-15686) by PCR and cloned into the petSUMO vector (Invitrogen).

All proteins were expressed and purified from the BL21 (DE3) strain of E. coli (New England Biolabs), under the induction of 1 mmol/L isopropyl 3-D-1-thiogalactopyranoside (IPTG), for 2 hours at 30° C. WT SrtA and SrtA* (harboring mutations P94S/D160N/K196T) both lacking the membrane-spanning domain (residues 1-58) were expressed and purified as described by Ling and colleagues (Ling et al., J. Am. Chem. Soc. 2012, 134, 10749-10752). mPA-LPETGG-His6 (“LPETGG-His6” disclosed as SEQ ID NO: 44) was purified from the periplasm as previously described (Mechaly et al., mBio 2012, 3, pii:e00088-12; Miller et al., Biochemistry 1999, 38, 10432-10441).

mDT-LPETGG-His₆ (“LPETGG-His6” disclosed as SEQ ID NO: 44), G5-ZHER2 (“G5” disclosed as SEQ ID NO: 9), and G5-4D5 (“G5” disclosed as SEQ ID NO: 9) were expressed from the petSUMO vector (Invitrogen) as His6-SUMO fusions (“His6” disclosed as SEQ ID NO: 5). Cell pellets were lysed by sonication in lysis buffer (20 mmol/L Tris-HCl pH 8.0, 150 mmol/L NaCl, 10 mmol/L imidazole, 10 mg lysozyme, 2 mg DNAse I, supplemented with a Roche complete protease inhibitor). His-tagged proteins were bound to Ni2+-NTA resin, washed with wash buffer (20 mmol/L Tris-HCl pH 8.0, 150 mmol/L NaCl, and 20 mmol/L imidazole), and eluted with wash buffer supplemented with 250 mmol/L imidazole. The resulting purified proteins were exchanged into imidazole-free buffer (20 mmol/L Tris-HCl, pH 8.0 and 150 mmol/L NaCl) and cleaved by SUMO protease for 1 hour at room temperature to generate mDT-LPETGG-His6 (“LPETGG-His6” disclosed as SEQ ID NO: 44) and RBPs displaying free N-terminal oligoglycine peptides. G5-ZHER2 (“G5” disclosed as SEQ ID NO: 9) and G5-4D5 (“G5” disclosed as SEQ ID NO: 9) were freed from the His6-SUMO tag (“His6” disclosed as SEQ ID NO: 5) by Ni2+ affinity chromatography. mDT-LPETGG-His6 (“LPETGG-His6” disclosed as SEQ ID NO: 44) was separated from the His6-SUMO tag (“His6” disclosed as SEQ ID NO: 5) by size exclusion chromatography on a HiLoad 16/60 Superdex 75 prep grade column attached to an automated Akta purifier (GE Healthcare Biosciences).

mDT-LPETGG-His₆ (“LPETGG-His6” disclosed as SEQ ID NO: 44) or mPA-LPETGG-His6 (“LPETGG-His6” disclosed as SEQ ID NO: 44) (50 μmol/L) was incubated with an excess of either G5-ZHER2 (“G5” disclosed as SEQ ID NO: 9) or G5-4D5 (“G5” disclosed as SEQ ID NO: 9) (200 μmol/L). Reactions were catalyzed by 5 μmol/L WT SrtA or SrtA* in sortase reaction buffer (50 mmol/L Tris-HCl, 10 mmol/L CaCl2, 150 mmol/L NaCl pH 7.5) at room temperature.

mTx-RBP fusions were purified from 0.5 mL reactions by sequential Ni2+-NTA and size exclusion chromatography steps (FIG. 1A). Ni2+-NTA resin (250 μL) was added to the ligation reactions to bind the His6-tagged (SEQ ID NO: 5) unreacted mTx substrate and SrtA* enzyme. The flow-through fraction was collected, and the resin was washed with an additional 1 mL of wash buffer. The flow-through and wash fractions were pooled and mTx-RBP fusions were separated from unreacted RBP using a HiLoad 16/60 Superdex 200 prep-grade size exclusion chromatography column.

Cells were plated in appropriate medium at densities of 3 to 3.5×10⁴ cells per well in 96-well plates and incubated overnight at 37° C. The following day, cells were exposed to medium supplemented with the toxin conjugate or toxin mixture. For mDT-variants, cells were exposed to eight 10-fold serial dilutions (starting with a final concentration of 100 nmol/L) for 24 hours. For mPA-variants, cells were exposed to 20 nmol/L mPA-ZHER2 ormPA-4D5 plus a 10-fold serial dilution of LF_{N}-DTA (starting with a final concentration of 100 nmol/L) for 4 hours. After the incubation period, toxin-containing medium was removed and replaced with leucine-deficient medium supplemented with 1 μCi of [³H]-leucine/mL (PerkinElmer) and incubated for an additional hour. Plates were washed twice with cold PBS (200 μL) before the addition of 200 μL of scintillation fluid. The amount of [³H]-leucine incorporated was determined by scintillation counting using a Wallac MicroBeta TriLux 1450 LSC (PerkinElmer). Percentage protein synthesis was normalized against untreated cells and plotted versus the concentration of LF_{N}-DTA or diphtheria toxin-variant in GraphPad Prism.

Competition assays were conducted as described earlier in which increasing concentrations of free G5-ZHER2 (“G5” disclosed as SEQ ID NO: 9) or G5-4D5 (“G5” disclosed as SEQ ID NO: 9) were added to medium containing 20 nmol/L mPA-ZHER2/mPA-4D5 plus LFN-DTA (1 nmol/L) and exposed to BT-474 cells for 4 hours. Percentage protein synthesis was normalized against untreated cells and plotted using GraphPad Prism.Cancer cell lines were seeded (3.5×10⁴ cells/well) in partitioned sections of a chambered tissue culture slide (Thermo Scientific). After an overnight incubation, the medium was removed, and the partitioning element was discarded. The slides were washed with PBS and incubated for 24 hours with RPMI medium containing (i) 20 nmol/L of mPA-ZHER2 with 10 nmol/L LF_{N}-DTA, (ii) 20 nmol/L mPA-4D5 plus 10 nmol/L LF_{N}-DTA, (iii) 100 nmol/L mDT-ZHER2, or (iv) 100 nmol/L mDT-4D5. Following toxin exposure, cells were processed as previously described (McCluskey et al., Mol. Oncol. 2013, 7, 440-451).

Results and Discussion.

HER2 is overexpressed in several cancers (Arteaga et al., Nat. Rev. Clin. Oncol. 2012, 9, 16-32; Berchuck et al., Cancer Res. 1990, 50, 4087-4091; Slamon et al., Science 1989, 244, 707-712; Gravalos and Jimeno, Ann. Oncol. 2008, 19, 1523-1529) and is the target of U.S. Food and Drug Administration (FDA)-approved protein therapeutics (e.g., trastuzumab and T-DM1), as well as receptor-redirected protein toxins in preclinical stages (McCluskey et al, Mol. Oncol. 2013, 7, 440-451; Cao et al., Mol. Cancer Ther. 2013, 12, 979-991; Cao et al., Cancer Res. 2009, 69, 8987-8995; Zielinski et al., Clin. Cancer Res. 2011, 17, 5071-5081). Some classes of toxins, such as diphtheria toxin and anthrax toxin, have evolved an active mechanism of crossing the endosomal membrane and delivering bioactive proteins to the cytosol (Collier Mol. Aspect Med. 2009, 30, 413-422). This endosomal escape mechanism can be exploited to deliver a cytocidal enzymatic “payload,” such as the catalytic domain of diphtheria toxin (DTA; Collier and Cole, Science 1969, 164, 1179-1181; Collier, J. Mol. Bio. 1967, 25, 83-98) used in the current work, or other bioactive polypeptides that modulate intracellular processes.

Ploegh and others have shown the use of SrtA in vitro to incorporate polypeptides (Kobashigawa et al., J Biomol NMR 2009, 43:145-50; Levary et al., PLoS ONE 2011, 6:e18342; Mao et al., J Am Chem Soc 2004, 126:2670-1), biochemical handles (e.g., biotin; Popp et al., Nat Chem Biol 2007, 3: 707-8), fluorescent probes (Popp et al., Nat Chem Biol 2007, 3: 707-8; Antos et al., J Am Chem Soc 2009, 131:10800-1), peptide nucleic acids (Pritz et al., J Org Chem 2007; 72:3909-12), sugars (Samantaray et al., J Am Chem Soc 2008; 130: 2132-3), lipids (Antos et al., J Am Chem Soc 2008; 130:16338-43), unnatural amino acids (Mao et al., J Am Chem Soc 2004; 126:2670-1), and chemical groups (Ling et al., J Am Chem Soc 2012; 134: 10749-52) into a number of structurally distinct proteins. Although SrtA can be expressed in E. coli and purified as a soluble enzyme (Ton-That et al., Proc Natl Acad Sci USA 1999, 96:12424-9; Ilangovan et al. Proc Natl Acad Sci USA 2001, 98:6056-61), its use for in vitro protein engineering has been limited by long reaction times (typically 16-24 hours) and the need for large quantities of enzyme (more than 30 μmol/L) to circumvent suboptimal kinetics [kcat/Km_{LPETG}=100-200 (mol/L)⁻¹ s⁻¹; Chen et al., Proc Natl Acad Sci USA 2011, 108:11399-404].

Recently, Chen and colleagues evolved SrtA by yeast display to generate mutants with improved kinetics (Chen et al., Proc Natl Acad Sci USA 2011, 108:11399-404). Here, we describe the use of WT SrtA and an evolved SrtA variant (SrtA*) to assemble receptor-directed chimeric protein toxins in vitro (FIG. 1). The approach requires two building blocks: (i) a mutated, receptor recognition-deficient toxin protein (mTx) containing a canonical C-terminal SrtA recognition motif (here, LPETGG (SEQ ID NO: 2)), and (ii) a heterologous RBP carrying an N-terminal oligoglycine peptide (FIG. 1). SrtA catalyzes cleavage of the toxin moiety between Thr and Gly of the recognition peptide and formation of a covalent bond between the carboxyl group of Thr and the amino group of the oligoglycine peptide of the RBP (FIG. 1; Ton-That et al., Proc Natl Acad Sci USA 1999, 96:12424-9; Kruger et al., Biochemistry 2004, 43:1541-51).

Two HER2-directed single-chain toxins were created by fusing mDT with a HER2-specific Affibody (ZHER2; Orlova et al., Cancer Res 2006, 66:4339-48) or humanized a single-chain antibody fragment (4D5; Carter et al., Proc Natl Acad Sci USA 1992, 89:4285-9); the products were designated mDT-ZHER2 and mDT-4D5, respectively (FIG. 1). The catalytic DTA chain contained within these single-chain toxins served as a cytocidal payload that causes inhibition of protein synthesis and apoptotic cell death upon its delivery to the cytosolic compartment of sensitive cells (Collier and Cole, Science 1969, 164, 1179-1181; Collier, J. Mol. Bio. 1967, 25, 83-98).

We also created two HER2-directed binary toxins. First, we fused ZHER2 or4D5 to the C-terminus of mPA (Mechaly et al., mBio 2012, 3, pii:e00088-12; Rosovitz et al., J. Biol. Chem. 2003, 278, 30936-30944), yielding mPA-ZHER2 and mPA-4D5 (FIG. 1). Protective antigen, the receptor-binding pore-forming component of anthrax toxin, noncovalently binds the enzymatic components of the toxin and delivers them to the cytosol (Cunningham et al., Proc Natl Acad Sci USA 2002; 99:7049-53; Mogridge et al., Proc Natl Acad Sci USA 2002, 99:7045-8). However, the effector moieties of anthrax toxin are not cytocidal toward most cell types, therefore we combined mPA-ZHER2 or mPA-4D5 with LF_{N}-DTA, an effector protein containing the high affinity N-terminal protective antigen-binding domain of the anthrax lethal factor (LF_{N}) with DTA. LF_{N}-DTA binds to mPA-ZHER2 and mPA-4D5 and upon its delivery to the cytosol, the DTA moiety blocks protein synthesis, as with the single-chain toxins. We also showed that using the same panel of cell lines, DTA delivery by recombinantly fused mPA-ZHER2 resulted in rapid protein synthesis inhibition and subsequent cell death via apoptosis (McCluskey et al., Mol Oncol 2013, 7:440-51). We used protein-synthesis inhibition as a sensitive readout for delivery of DTA to the cytosol to monitor the functions of the SrtA fusions. DTA thus served as the enzymatic effector moiety of all of the targeted toxins in our study.

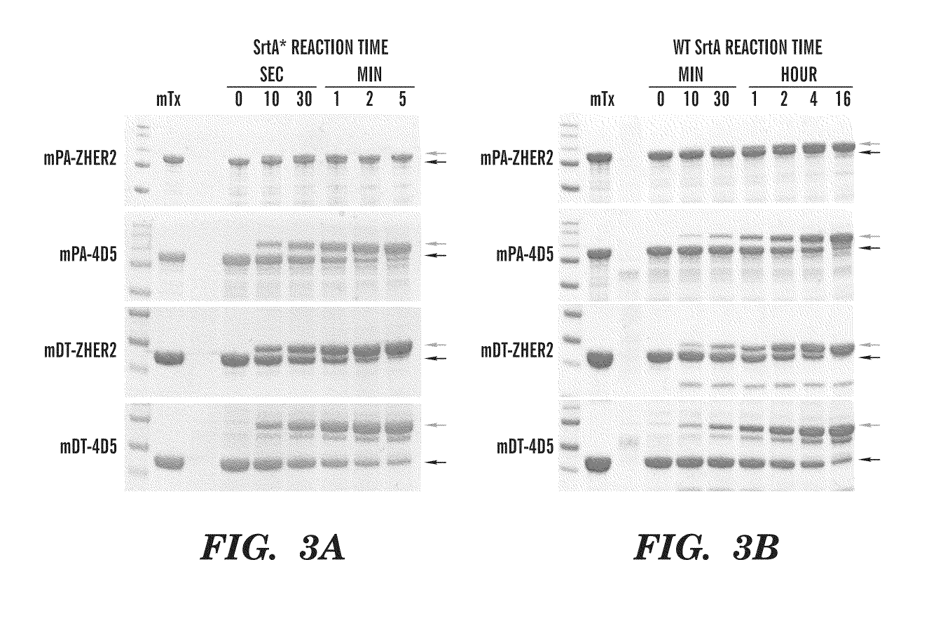

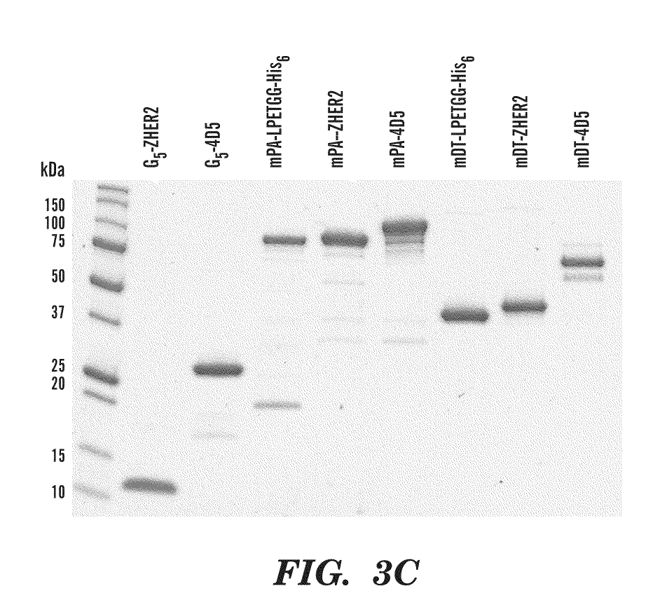

The fusion reaction creating mDT-ZHER2, mDT-4D5, mPA-ZHER2, or mPA-4D5 was virtually complete within 5 minutes when 5 mmol/L SrtA* was used as catalyst, whereas the same concentration of WT SrtA required more than 4 hours to achieve the same level of fusion (compare FIGS. 3A and 3B). Reaction rates showed no significant dependence on specific substrate proteins, indicating that the nature of the folded polypeptide entities to which the LPETGG (SEQ ID NO: 2) and G5 (SEQ ID NO: 9) tags were attached mattered little in the Srt-catalyzed reactions. SrtA* reaction rates were consistent with results reported by Ling and colleagues to ligate peptides with chemical groups to proteins for use in semi-synthesis strategies (Ling et al., J Am Chem Soc 2012, 134: 10749-52).

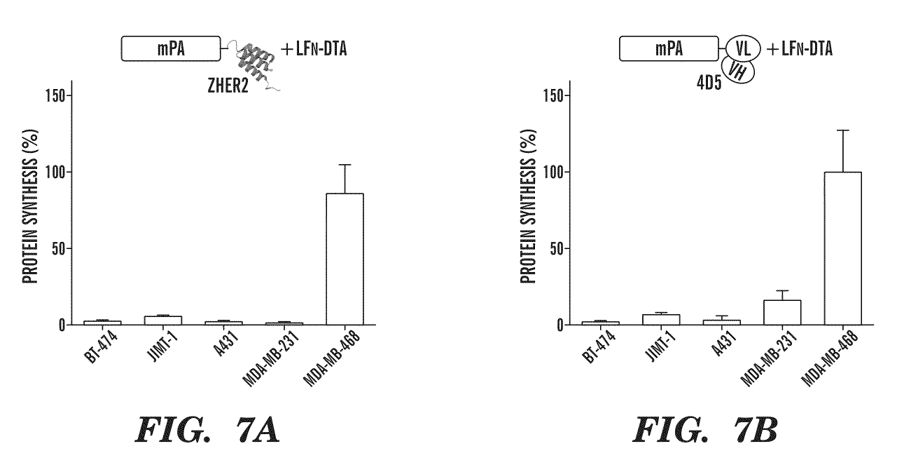

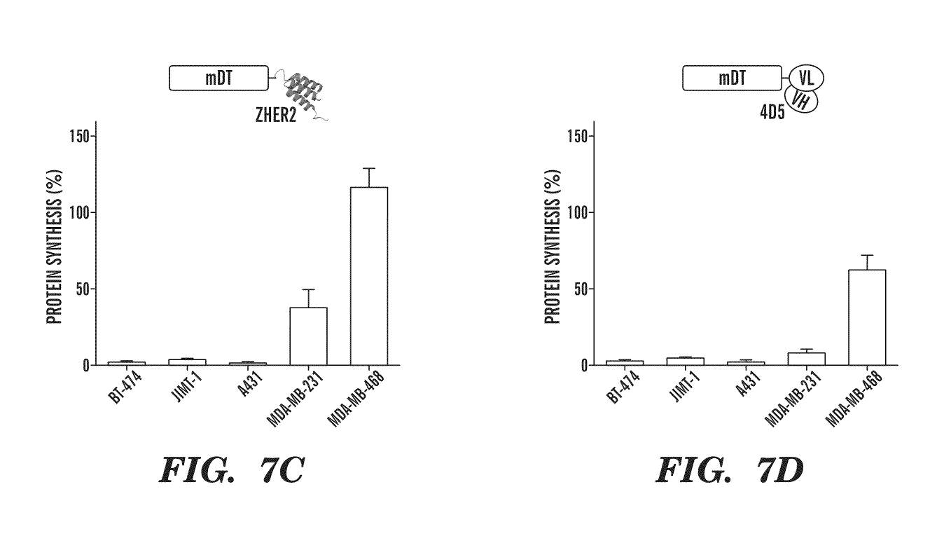

SrtA*-ligated fusions were purified by sequential Ni²⁺-NTA and size exclusion chromatography steps to give virtually pure products (yield 20%-65%; FIG. 3C; Table 3). Cancer cell lines expressing various levels of HER2, including a trastuzumab-resistant line isolated from a HER2-positive patient clinically resistant to tras-tuzumab (Tanner et al., Mol Cancer Ther 2004, 3: 1585-92), were incubated with either mPA-ZHER2/mPA-4D5 plus LF_{N}-DTA for 4 hours or mDT-ZHER2/mDT-4D5 for 24 hours. Following toxin exposure, protein synthesis was measured over a 1-hour period. All four fusions were able to direct toxin action to HER2-positive cells, and the degree of cell killing was dependent on the level of cell-surface HER2 (FIGS. 4A, 4B, 5A and 5B; McCluskey et al., Mol Oncol 2013, 7:440-51; Rusnak et al., Cell Prolif 2007, 40:580-94). Cells expressing the highest levels were the most sensitive (BT-474; FIGS. 4A, 4B, 5A and 5B, Table 3), and HER2-negative cells, MDA-MB-468, were unaffected (FIGS. 4A, 4B, 5A and 5B). The specificity of SrtA*-generated toxin fusions for cells bearing the cognate receptor was confirmed, and the absence of bystander effects on cells lacking HER2 shown, in experiments conducted with mixed cell populations of HER2-positive and -negative cells (FIG. 7A to 7D). Some non-specific toxicity was observed for mDT-4D5 toward HER2-negative MDA-MB-468 cells in mixed cell populations. Unlike mPA and LF_{N}-DTA, mDT retains some residual nonspecific activity toward even toxin-resistant cells (Pappenheimer et al., J Infect Dis 1982, 145:94-102), an effect that is reduced when the protein is attached to an RBP. It should be noted that the nonspecific toxicity is insignificant, and does not in any capacity impact the use of mDT fusions in clinical applications. For example, a doctor/clinician can reduce the dose of mDT fusions that are administered to minimize nonspecific toxicity. The decrease in protein synthesis may indicate that 4D5, when fused to mDT, does not have the same steric effects as ZHER2 in shielding off-target toxicity of mDT in mixed populations.

TABLE 3
SrtA* ligation reaction yields and in vitro activities
of HER2-targeted toxin fusions on various cell lines

| | | Cell line EC₅₀ (mol/L)^{a} | | | | |
|---|---|---|---|---|---|---|
| | | | | | MDA- | MDA- |
| Toxin | % Yield^{b} | BT-474 | JIMT-1 | A431 | MB-231 | MB-468 |
| mPA- | 35 ± 6 | 9.1 × 10⁻¹³ | 2.3 × 10⁻¹² | 2.3 × 10⁻¹¹ | 6 × 10⁻¹⁰ | >1 × 10⁻⁷ |
| ZHER2 + LF_{N}- | | | | | | |
| DTA^{c} | | | | | | |
| mPA- | 20 ± 3 | 1.5 × 10⁻¹² | 1.6 × 10⁻¹¹ | 8.6 × 10⁻¹¹ | 2.2 × 10⁻⁹ | >1 × 10⁻⁷ |
| 4D5 + LF_{N}- | | | | | | |
| DTA^{c} | | | | | | |
| mDT- | 67 ± 4 | 1.7 × 10⁻¹¹ | 1.2 × 10⁻⁹ | 5.4 × 10⁻⁹ | >1 × 10⁻⁷ | >1 × 10⁻⁷ |
| ZHER2^{d} | | | | | | |
| mDT-4D5 | 38 ± 9 | 1.3 × 10⁻¹¹ | 5.9 × 10⁻¹⁰ | 3.0 × 10⁻⁹ | 8.3 × 10⁻⁸ | >1 × 10⁻⁷ |

^{a}EC₅₀ values were calculated in GraphPad Prism from dose-response curves (presented in FIGS. 4A-4B and 5A-5B).
^{b}Molar ratio of the mTx-RBP product versus mTx after purification. Average of three independent reactions.
^{c}Measured by [³H]-leucine incorporation after 4-hour toxin exposure.
^{d}Measured by [³H]-leucine incorporation after 24-hour toxin exposure.

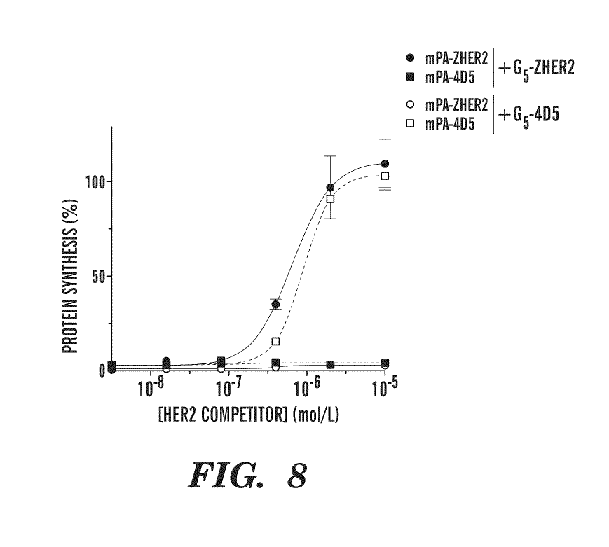

Free ZHER2 Affibody competitively inhibited mPA-ZHER2-dependent cell killing, but not mPA-4D5-dependent killing (FIG. 8); and free 4D5 protected cells against mPA-4D5 plus LF_{N}-DTA, but not against mPA-ZHER2-dependent killing under the same conditions (FIG. 8). These findings are consistent with structural data showing ZHER2 and a Fab fragment of trastuzumab (from which 4D5 is derived) recognize nonoverlapping HER2 epitopes (Pappenheimer et al., J Infect Dis 1982, 145:94-102).

While both the mPA fusions and the mDT fusions were found to be surprisingly potent, the combinations of mPA-ZHER2 or mPA-4D5 with LF_{N}-DTA were 10 to 100-fold more potent than the corresponding diphtheria toxin fusions (Table 3), as the mPA chimeric toxins acted in a shorter incubation period (4 hours vs. 24 hours) and were able to kill MDA-MB-231, a cell line expressing low levels of HER2 (FIGS. 4A, 4B, 5A and 5B). The difference in EC₅₀ for the toxin conjugates could be a result of more efficient effector delivery by mPA, combined with its ability to deliver multiple enzymatic effectors (Mogridge et al., Biochemistry 2002, 41:1079-82).

The results we report here have shown the use of Srt-based fusion to modify the receptor specificity of both a single-chain and a binary toxin. Each of two structurally diverse toxins was fused to two equally diverse HER2-binding proteins, underlining the versatility of SrtA-based protein fusion and its practical use for fusing a broad array of appropriately tagged proteins, for example, DTA to cholera holotoxin (Guimaraes et al., J Cell Biol 2011, 195, 751-764). The fact that fusion reactions in our study surprisingly reached completion within a few minutes when SrtA* was used makes our evolved form of SrtA particularly attractive in real life applications.

We claim:

1. A method for making a protein toxin comprising a receptor-binding ligand, the method comprising the steps of:
a. providing a protein toxin substrate and a C-terminal sortase-recognition motif optionally followed by an affinity epitope that is separated from the toxin by a linker;
b. providing a targeting moiety comprising an N-terminal peptide; and
c. contacting the protein toxin substrate of step (a) with the targeting moiety of step (b) with a sortase enzyme.

2. The method of claim 1, wherein the protein toxin substrate does not comprise its natural receptor binding domain or comprises a non-functional natural receptor binding domain.

3. The method of claim 1, wherein the targeting moiety is a receptor targeting ligand.

4. The method of claim 1, wherein the receptor is HER 2.

5. The method of claim 3, wherein the receptor targeting ligand is a HER2 antibody or HER2 AFFIBODY.

6. The method of claim 1, wherein the C-terminal sortase recognition motif is a sortase A recognition motif.

7. The method of claim 6, wherein the sortase A recognition motif is LPXTG (SEQ ID NO: 1), wherein X is any amino acid.

8. The method of claim 7, wherein the sortase A recognition motif is LPETGG (SEQ ID NO: 2).

9. The method of claims 1, wherein the C-terminal sortase recognition motif is a sortase B recognition motif.

10. The method of claim 9, wherein the sortase B recognition motif is NPQTN (SEQ ID NO: 3) or NPKTG (SEQ ID NO: 4).

11. The method of claim 1, wherein the affinity epitope is selected from a Histidine repeat (His₆) (SEQ ID NO: 5), maltose binding protein (MBP), protein A (ProtA), glutathione S-transferase (GST), calmodulin binding peptide (CBP), calmodulin, thioredoxin, Strep-tags, hemagglutinin, biotin, FLAG, V5, and c-myc.

12. The method of claim 1, wherein the linker comprises at least one Glycine-Serine repeat.

13. The method of claim 12, wherein the linker comprises 1-10 Glycine-Serine repeats (SEQ ID NO: 6).

14. The method of claim 13, wherein the linker comprises 3 or 4 Glycine-Serine repeats (SEQ ID NO: 7).

15. The method of claim 1, wherein the N-terminal peptide consists of more than two Glycine residues.

16. The method of claim 15, wherein the N-terminal peptide consists of 3-10 Glycine residues (SEQ ID NO: 8).

17. The method of claim 16, wherein the N-terminal peptide consists of five Glycine residues (SEQ ID NO: 9).

18. The method of claim 1, wherein the protein toxin is an anthrax toxin.

19. The method of claim 1, wherein the protein toxin is a diphtheria toxin.

20. The method of claim 1, further comprising the step of purifying the toxin that comprises a receptor-binding ligand.

21. The method of claim 20, wherein the step of purifying comprises sequential Ni²⁺-NTA affinity and size exclusion chromatography.


SEQUENCE LISTING (47 sequences)

SEQ ID NO: 1 - protein, 5 aa, Artificial Sequence
  Description of Artificial Sequence Synthetic peptide
LPXTG

SEQ ID NO: 2 - protein, 6 aa, Artificial Sequence
  Description of Artificial Sequence Synthetic peptide
LPETGG

SEQ ID NO: 3 - protein, 5 aa, Artificial Sequence
  Description of Artificial Sequence Synthetic peptide
NPQTN

SEQ ID NO: 4 - protein, 5 aa, Artificial Sequence
  Description of Artificial Sequence Synthetic peptide
NPKTG

SEQ ID NO: 5 - protein, 6 aa, Artificial Sequence
  Description of Artificial Sequence Synthetic 6xHis tag
HHHHHH

SEQ ID NO: 6 - protein, 20 aa, Artificial Sequence
  Description of Artificial Sequence Synthetic peptide
GSGSGSGSGSGSGSGSGSGS

SEQ ID NO: 7 - protein, 8 aa, Artificial Sequence
  Description of Artificial Sequence Synthetic peptide
GSGSGSGS

SEQ ID NO: 8 - protein, 10 aa, Artificial Sequence
  Description of Artificial Sequence Synthetic peptide
GGGGGGGGGG

SEQ ID NO: 9 - protein, 5 aa, Artificial Sequence
  Description of Artificial Sequence Synthetic peptide
GGGGG

SEQ ID NO: 10 - protein, 6 aa, Artificial Sequence
  Description of Artificial Sequence Synthetic peptide
GSGSGS

SEQ ID NO: 11 - protein, 8 aa, Artificial Sequence
  Description of Artificial Sequence Synthetic peptide
GSGSGSGS

SEQ ID NO: 12 - protein, 4 aa, Artificial Sequence
  Description of Artificial Sequence Synthetic peptide
KDEL

SEQ ID NO: 13 - protein, 8 aa, Artificial Sequence
  Description of Artificial Sequence Synthetic peptide
GGHHHHHH

SEQ ID NO: 14 - protein, 5 aa, Artificial Sequence
  Description of Artificial Sequence Synthetic peptide
LPSTG

SEQ ID NO: 15 - protein, 8 aa, Artificial Sequence
  Description of Artificial Sequence Synthetic peptide
DYKDDDDK

SEQ ID NO: 16 - protein, 10 aa, Artificial Sequence
  Description of Artificial Sequence Synthetic peptide
HHHHHHHHHH

SEQ ID NO: 17 - protein, 10 aa, Artificial Sequence
  Description of Artificial Sequence Synthetic peptide
EQKLISEEDL

SEQ ID NO: 18 - protein, 9 aa, Artificial Sequence
  Description of Artificial Sequence Synthetic peptide
YPYDVPDYA

SEQ ID NO: 19 - protein, 11 aa, Artificial Sequence
  Description of Artificial Sequence Synthetic peptide
YTDIEMNRLGK

SEQ ID NO: 20 - protein, 11 aa, Artificial Sequence
  Description of Artificial Sequence Synthetic peptide
QPELAPEDPED

SEQ ID NO: 21 - protein, 14 aa, Artificial Sequence
  Description of Artificial Sequence Synthetic peptide
GKPIPNPLLGLDST

SEQ ID NO: 22 - protein, 15 aa, Artificial Sequence
  Description of Artificial Sequence Synthetic peptide
GLNDIFEAQKIEWHE

SEQ ID NO: 23 - protein, 26 aa, Artificial Sequence
  Description of Artificial Sequence Synthetic peptide
KRRWKKNFIAVSAANRFKKISSSGAL

SEQ ID NO: 24 - protein, 15 aa, Artificial Sequence
  Description of Artificial Sequence Synthetic peptide
KETAAAKFERQHMDS

SEQ ID NO: 25 - protein, 38 aa, Artificial Sequence
  Description of Artificial Sequence Synthetic polypeptide
MDEKTTGWRGGHVVEGLAGELEQLRARLEHHPQGQREP

SEQ ID NO: 26 - protein, 13 aa, Artificial Sequence
  Description of Artificial Sequence Synthetic peptide
SLAELLNAGLGGS

SEQ ID NO: 27 - protein, 8 aa, Artificial Sequence
  Description of Artificial Sequence Synthetic peptide
DLYDDDDK

SEQ ID NO: 28 - protein, 16 aa, Artificial Sequence
  Description of Artificial Sequence Synthetic peptide
TDKDMTITFTNKKDAE

SEQ ID NO: 29 - protein, 13 aa, Artificial Sequence
  Description of Artificial Sequence Synthetic peptide
AHIVMVDAYKPTK

SEQ ID NO: 30 - protein, 8 aa, Artificial Sequence
  Description of Artificial Sequence Synthetic peptide
WSHPQFEK

SEQ ID NO: 31 - protein, 30 aa, Artificial Sequence
  Description of Artificial Sequence Synthetic polypeptide
GSGSGSGSGSGSGSGSGSGSGSGSGSGSGS

SEQ ID NO: 32 - protein, 40 aa, Artificial Sequence
  Description of Artificial Sequence Synthetic polypeptide
GSGSGSGSGSGSGSGSGSGSGSGSGSGSGSGSGSGSGSGS

SEQ ID NO: 33 - protein, 30 aa, Artificial Sequence; identical to SEQ ID NO: 31
  Description of Artificial Sequence Synthetic polypeptide

SEQ ID NO: 34 - protein, 20 aa, Artificial Sequence
  Description of Artificial Sequence Synthetic peptide
GSGSGSGSGSGSGSGSGSGS

SEQ ID NO: 35 - protein, 10 aa, Artificial Sequence
  Description of Artificial Sequence Synthetic peptide
GSGSGSGSGS

SEQ ID NO: 36 - protein, 20 aa, Artificial Sequence
  Description of Artificial Sequence Synthetic polypeptide
GGGGGGGGGGGGGGGGGGGG

SEQ ID NO: 37 - protein, 15 aa, Artificial Sequence
  Description of Artificial Sequence Synthetic polypeptide
GGGGGGGGGGGGGGG

SEQ ID NO: 38 - protein, 10 aa, Artificial Sequence
  Description of Artificial Sequence Synthetic peptide
GGGGGGGGGG

SEQ ID NO: 39 - protein, 735 aa, Artificial Sequence
  Description of Artificial Sequence Synthetic polypeptide
EVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYAAKLPLYISNPNYKVNVYAVTKENTIINPSENGDTSTNGIKKILIFSKKGYEIG

SEQ ID NO: 40 - protein, 58 aa, Artificial Sequence
  Description of Artificial Sequence Synthetic polypeptide
VDNKFNKEMRNAYWEIALLPNLNNQQKRAFIRSLYDDPSQSANLLAEAKKLNDAQAPK

SEQ ID NO: 41 - protein, 252 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTPSHNSHQVPSAGGPTANSGTSGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 42 - protein, 386 aa, Artificial Sequence
  Description of Artificial Sequence Synthetic polypeptide
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVSYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKT

SEQ ID NO: 43 - protein, 344 aa, Artificial Sequence
  Description of Artificial Sequence Synthetic polypeptide
GGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 44 - protein, 12 aa, Artificial Sequence
  Description of Artificial Sequence Synthetic peptide
LPETGGHHHHHH

SEQ ID NO: 45 - protein, 18 aa, Artificial Sequence
  Description of Artificial Sequence Synthetic peptide
GSGSGSLPETGGHHHHHH

SEQ ID NO: 46 - protein, 15 aa, Artificial Sequence
  Description of Artificial Sequence Synthetic peptide
GSGSGSLPETGGGGG

SEQ ID NO: 47 - protein, 15 aa, Artificial Sequence
  Description of Artificial Sequence Synthetic polypeptide
GGGGGGGGGGGGGGG